(19) United States
(12) Patent Application Publication
(10) Pub. No.: US 2026/0072020 A1
(43) Pub. Date: Mar. 12, 2026

(54) PRINTABLE MOLECULE-SELECTIVE CORE-SHELL NANOPARTICLES FOR WEARABLE AND IMPLANTABLE SENSING

(71) Applicant: CALIFORNIA INSTITUTE OF TECHNOLOGY, Pasadena, CA (US)

(72) Inventors: Wei GAO, Pasadena, CA (US); Minqiang Wang, Pasadena, CA (US)

(21) Appl. No.: 19/322,496

(22) Filed: Sep. 8, 2025

Related U.S. Application Data
(60) Provisional application No. 63/691,539, filed on Sep. 6, 2024.

(51) Int. Cl. G01N 33/543; B22F 1/054; B22F 1/102; B22F 1/145

(52) U.S. Cl. CPC G01N 33/54346; B22F 1/0553; B22F 1/102; B22F 1/145; B22F 2201/02; B22F 2301/15; G01N 2600/00

(57) ABSTRACT

Printable, molecule-selective core-shell nanoparticles that couple a redox-active core with a molecularly imprinted polymer (MIP) shell. The core may comprise nickel hexacyanoferrate nanocubes with improved redox stability in physiological media. A thin MIP shell may be formed by templated copolymerization (e.g., methacrylic acid with ethylene glycol dimethacrylate) around the nanocubes, followed by template extraction to generate target-complementary binding cavities. Monomer selection may be guided computationally to maximize binding energy and selectively for a chosen analyte. Target binding within the MIP shell may modulate interfacial electron transfer at the core, enabling more robust and reversible electrochemical transduction. The nanoparticles may be formulated into stable, inkjet-printable dispersions via optimized solvent systems and exhibit cytocompatibility, anti-biofouling behavior, thermal resilience, and long room-temperature shelf stability.

### CROSS-REFERENCE TO RELATED APPLICATIONS

This application claims the benefit of U.S. Provisional Application No. 63/691,539 filed on Sep. 6, 2024, the contents of which are incorporated herein by reference in their entirety.

### STATEMENT REGARDING FEDERALLY SPONSORED R&D

This invention was made with government support under Grant No. HL155815 awarded by the National Institutes of Health and under Grant No. 2145802 awarded by the National Science Foundation. The government has certain rights in the invention.

### TECHNICAL FIELD

The present disclosure relates generally to printable core-shell nanoparticles, and in some embodiments, may relate to nanoparticles with a redox-active Prussian Blue analogue core and a molecularly imprinted polymer shell for target-selective molecular recognition and electrochemical transduction.

### BACKGROUND

There is sustained interest in materials and devices capable of selectively detecting small molecules relevant to health monitoring, diagnostics, and therapy management. Existing biorecognition strategies that rely on biomacromolecules (e.g., enzymes, antibodies, aptamers) can offer specificity but may exhibit limited stability, susceptibility to denaturation or leaching, and batch-to-batch variability, particularly in complex biofluids and under variable environmental conditions. Synthetic recognition approaches, including templated polymers that form complementary binding sites, have been investigated to address some of these limitations.

Electrochemical techniques are sometimes used for translating molecular interactions into measurable signals, and diverse redox-active materials and mediators have been employed for this purpose. However, long-term operation in physiologically relevant media can present challenges such as signal drift, material degradation, fouling, and reduced reproducibility. Nanoscale constructs, including particles with tailored surfaces and architectures, have been explored to enhance surface area, tunability, and integration into sensing formats. Separately, printed and additive manufacturing methods are conventionally used for producing conductive features and functional layers on flexible substrates.

Despite these developments, recognized needs remain for robust, selective, and stable recognition materials that maintain performance during prolonged exposure to biofluids, resist fouling, support regeneration, and are compatible with high-throughput, low-cost, and consistent manufacturing processes suitable for broad deployment.

### BRIEF SUMMARY

Various embodiments of the present disclosure may discuss nanoparticles comprising a redox-active core and a shell disposed on an outer surface of the redox-active core, the shell may comprise a plurality of target-specific binding cavities. In some embodiments the shell may be a molecularly imprinted polymer comprising a monomer and a crosslinker (e.g., a methacrylic acid monomer and an ethylene glycol dimethacrylate crosslinker), and the target-specific binding cavities may be formed by polymerizing one or more monomers in the presence of a template molecule and an initiator, and subsequently removing the template molecule (template molecules may include ascorbic acid, tryptophan, creatinine, busulfan, cyclophosphamide, mycophenolic acid, glutamate, phenylalanine, cortisol, dopamine, bisphenol A, aflatoxin B1, glucose, uric acid, lactate, COVID-19 spike protein fragments, microRNAs, etc.). The redox-active core can comprise a Prussian blue analogue such as NiHCF, CoHCF, CuHCF, FeHCF, Prussian Blue, MnHCF, ZnHCF, VHCF, CrHCF, MB modified nanoparticles, Fc and derivative nanoparticles, Azure A/C modified nanoparticles, toluidine blue O modified nanoparticles, [Ru(NH₃)₆]^{3+/2+}, [Os(bpy)₃]^{2+/3+}, cobaltocene, and other molecularly relevant materials. In some embodiments the core may comprise nickel hexacyanoferrate nanocubes.

Further embodiments may discuss a nanoparticle with a core comprising nanocubes and a molecularly imprinted polymer shell disposed on the nanocubes, the molecularly imprinted shell may comprise a cross-linked copolymer formed in the presence of a template molecule and subjected to template extraction to generate target-selective binding cavities that are complementary in shape, size, and orientation to the template molecule. The core can be a redox-active core and the nanocubes can comprise nickel hexacyanoferrate or a Prussian blue analogue. The cross-linked copolymer can be formed by co-polymerization of methacrylic acid and ethylene glycol dimethacrylate, or other relevant materials.

Further embodiments may discuss a method of manufacturing nanoparticles by synthesizing a redox-active core comprising nanocubes by reacting compounds in the presence of a chelating agent, contacting the nanocubes with a mixture of a monomer, a crosslinker, a template molecule, and an initiator, polymerizing the mixture to form a molecularly imprinted polymer shell on the nanocubes, and extracting the template molecule from the molecularly imprinted polymer shell to yield nanoparticles comprising target-selective binding cavities.

In various embodiments, the nanocubes may be nickel hexacyanoferrate nanocubes, the compounds may comprise nickel (II) salt and potassium hexacyanoferrate (III); the chelating agent may be trisodium citrate dihydrate, EDTA, oxalic acid, tartaric acid, ethylenediamine, triethanolamine, citric acid, ascorbic acid, glutathione, thiol group chelates heavy metals (Au, Cd), nitrilotriacetic acid, Ni²⁺/Co²⁺, cyanide, Fe(CN)₆]^{3-/4-}, 8-hydroxyquinoline, and dithiocarbamates; the monomer may be methacrylic acid (MAA), acrylamide (ACM), 4-vinylbenzoic acid (4VB), acrylic acid (AA), itaconic acid, 2-vinylpyridine (2-VP), N-vinylpyrrolidone (NVP), styrene, divinylbenzene (DVB), ethylene glycol dimethacrylate (EGDMA), trimethylolpropane trimethacrylate (TRIM), N,N′-methylenebisacrylamide (MBA), dopamine methacrylamide, boronic acid monomers, 3-acrylamidophenylboronic acid, poly(ethylene glycol) diacrylate (PEGDA), phosphorylcholine methacrylate, N-isopropylacrylamide (NIPAM), and/or spironaphthoxazine methacrylate; the cross linker may be EGDMA, DVB, TRIM, PETA, PEDGA, Bis-EMA, MBA, DATD, GPTMS, TEOS, chitosan-glutaraldehyde, genipin, DSBA, azobenzene dimethacrylate, boronates, 4-vinylphenylboronic acid, metal-organic crosslinkers, and/or zirconium methacrylate; and the initiator may be azobisisobutyronitrile (AIBN), ammonium persulfate (APS), benzoyl peroxide (BPO), 2,2-dimethoxy-2-phylacetophenone (DMPA), irgacure 2959, eosin Y, tetramethylethylenediamine (APS/TEMED), ferrous sulfate, ferrocenium salts, anthraquinone derivatives, glucose oxidase, horseradish peroxidase (HRP), and/or biorthogonal. In other embodiments, other compounds may be used for each component.

Additional steps and conditions may include injecting an aqueous solution comprising nickel (II) acetate and trisodium citrate into an aqueous solution of potassium hexacyanoferrate at a first temperature over a first period of time, carrying polymerization at a second temperature for a second period of time under a nitrogen atmosphere, extracting the template molecule by washing with a solution comprising an organic solvent, and centrifuging and washing the nanoparticles three times with water followed by drying under vacuum.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein.

### BRIEF DESCRIPTION OF THE SEVERAL VIEWS OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a diagram showing a printable core-shell nanoparticle, in accordance with various embodiments of the disclosed technology.

FIG. 2 is a diagram showing an electrochemical sensing mechanism of a printable core-shell nanoparticle, in accordance with various embodiments of the disclosed technology.

FIG. 3 is a diagram showing a solution synthesis of a printable core-shell nanoparticle, in accordance with various embodiments of the disclosed technology.

FIG. 4 is a diagram showing a chelate-assisted synthesis of Prussian blue analogues as the core of a printable core-shell nanoparticle, in accordance with various embodiments of the disclosed technology.

FIG. 5A is a diagram showing an example flexible and wireless microfluidic wearable patch as an implementation of printable core-shell nanoparticles, in accordance with various embodiments of the disclosed technology.

FIG. 5B is a diagram showing an example layered design of a flexible and wireless microfluidic wearable patch as an implementation of printable core-shell nanoparticles, in accordance with various embodiments of the disclosed technology.

FIG. 6 is a diagram showing an example of mass-produced flexible and wireless microfluidic wearable patches and an example implementation thereof, in accordance with various embodiments of the disclosed technology.

FIG. 7A is a diagram showing exemplary imaging of printable core-shell nanoparticles, in accordance with various embodiments of the disclosed technology.

FIG. 7B is a diagram showing exemplary cyclic voltammogram oxidation peak current changes of various materials for printable core-shell nanoparticles, in accordance with various embodiments of the disclosed technology.

FIG. 7C is a diagram showing an exemplary ion intercalation mechanism for printable core-shell nanoparticles, in accordance with various embodiments of the disclosed technology.

FIG. 7D is a diagram showing an example of highest occupied molecular orbitals of solvent molecules used to disperse printable core-shell nanoparticles, in accordance with various embodiments of the disclosed technology.

The figures are not intended to be exhaustive or to limit the invention to the precise form disclosed. It should be understood that the invention can be practiced with modification and alteration, and the disclosed technology be limited only by the claims and the equivalents thereof.

### DETAILED DESCRIPTION

Embodiments of the present disclosure may discuss printable, molecule-selective core-shell nanoparticles that may integrate a redox-active core with a molecularly imprinted polymer (MIP) shell. In the discussed embodiments, the core furnishes a stable, reversible electrochemical transducer comprised within a crystalline or coordination-polymer framework, while the MIP shell presents target-specific binding cavities formed by templated polymerization and subsequent template removal. The resulting nanoparticle architectures may be engineered to convert target binding events into modulations of the core's redox signal, enabling selective, robust, and regenerable detection of a wide range of small-molecule analytes. These nanoparticles may be formulated as stable dispersions suitable for inkjet printing or other deposition techniques, allowing integration into diverse substrates and devices; sensor implementations may be discussed herein as exemplary uses of embodiments of the nanoparticles.

In certain embodiments, the redox-active core may comprise a Prussian blue analogue (PBA). For example, nickel hexacyanoferrate (NiHCF) may be used to construct the core and, in some embodiments, may be provided as uniform nanocubes produced by chelate-modulated aqueous synthesis. The nanocubes can exhibit substantially homogeneous elemental distributions and well-defined cubic morphology and can maintain crystallographic integrity and redox activity during prolonged operation in physiologically relevant media. Other embodiments may contemplate alternative PBAs and redox-active cores, including cobalt, copper, iron, manganese, zinc, vanadium, and chromium hexacyanoferrates; particles bearing covalently or coordinatively bound organic redox moieties (e.g., methylene blue, ferrocene derivatives, Azure dyes, toluidine blue O); and inorganic redox complexes (e.g., [Ru(NH3)6]3+/2+, [Os(bpy)3]2+/3+, cobaltocene). The core can be tailored in composition, size, and morphology to achieve desired redox potential, kinetics, and stability in the intended operating medium.

In exemplary embodiments, NiHCF nanocubes may be synthesized by reacting a nickel(II) salt with potassium hexacyanoferrate(III) in water in the presence of a chelating agent that moderates nucleation and growth to yield monodisperse particles. Trisodium citrate may be a representative chelating agent, and additional embodiments may employ other carboxylates, aminocarboxylates, polyamines, phosphates, or biomolecule-derived ligands (e.g., EDTA, oxalic acid, tartaric acid, ethylenediamine, triethanolamine, nitrilotriacetic acid, citric acid, ascorbic acid, glutathione, 8-hydroxyquinoline, dithiocarbamates). Process conditions can include controlled reagent addition (e.g., injection of a metal/citrate solution into hexacyanoferrate), room-temperature reaction over several hours, and post-synthesis washing and drying. In some embodiments, the synthesized cores may have characteristic edge lengths on the order of tens to a few hundreds of nanometers (e.g., about 100 nm), with particle size and dispersity regulated by reagent concentrations, chelator identity, and addition rate.

The MIP shell may be formed directly on the surface of the redox-active core by polymerizing one or more functional monomers with a crosslinker in the presence of a template molecule and an initiator. In certain embodiments, methacrylic acid may be a functional monomer and ethylene glycol dimethacrylate may be a crosslinker, with azobisisobutyronitrile (AIBN) initiating thermal free-radical polymerization. The core particles may be dispersed in a solvent system comprising the monomer(s), crosslinker, template, and initiator; after polymerization, the template may be extracted to reveal target-complementary binding cavities distributed throughout the thin shell. The shell can be engineered for affinity and selectivity to a chosen analyte by selecting monomers that present complementary noncovalent interaction motifs (e.g., hydrogen bonding, ionic interactions, I-I interactions, boronate-diol complexation, etc.) or other binding chemistries.

Additional embodiments may utilize alternative functional monomers (e.g., acrylamide, 4-vinylbenzoic acid, acrylic acid, itaconic acid, 2-vinylpyridine, N-vinylpyrrolidone, styrenic monomers, boronic-acid-containing monomers, phosphorylcholine methacrylate, N-isopropylacrylamide, etc.), alternative crosslinkers (e.g., divinylbenzene, trimethylolpropane trimethacrylate, pentaerythritol triacrylate, poly(ethylene glycol) diacrylate, Bis-EMA, N,N′-methylenebisacrylamide, diallyl tartardiamide, silane/sol-gel crosslinkers, genipin, redox- or photo-cleavable linkers, etc.), and alternative initiation modes (e.g., ammonium persulfate, benzoyl peroxide, photoinitiators such as DMPA or Irgacure 2959, visible-light systems such as eosin Y, redox pairs such as APS/TEMED, electrochemical initiation, or enzyme-mediated radical generation, etc.). Polymerization can be carried out under inert atmosphere at a controlled temperature and time to obtain a conformal, thin shell; template removal can be achieved with organic or mixed solvents (e.g., acetic acid/methanol) and repeated washing.

The molecular recognition and signal transduction mechanisms may result from the cooperative action of the MIP shell and the redox-active core. In operation, target molecules in a sample may bind within the shell's target-specific cavities, which may be complementary in shape, size, and chemical functionality to the template used during imprinting. Binding within the shell may modulate the interfacial ion/electron transport between the surrounding media and the confined redox-active core, producing a quantifiable change in the observable redox response of the core. In embodiments employing electrochemical readout, this modulation may be reflected in diminished redox peak currents or altered charge-transfer resistance upon target binding, and the magnitude of the change can correlate with analyte concentration. Because the mediator may be structurally part of, or confined within, the core, the architecture may reduce mediator leaching and support repeated use, including electrochemical regeneration to remove bound targets by applying a suitable potential.

Embodiments may support broad analyte scope by selecting appropriate templates and monomer chemistries. Representative template molecules may include metabolites and nutrients (e.g., ascorbic acid, tryptophan, creatinine, glucose, uric acid, lactate, amino acids, etc.), hormones and neurotransmitters (e.g., cortisol, dopamine, etc.), small-molecule drugs (e.g., cyclophosphamide, busulfan, mycophenolic acid, etc.), environmental and food-safety analytes (e.g., bisphenol A, aflatoxin B1, etc.), and emerging targets (e.g., viral protein fragments, microRNAs, etc.). In various embodiments, computational workflows can be employed to guide monomer selection and shell design by estimating binding energies and selectivity against interferents based on docking and quantum chemical calculations; such computationally predicted monomer-template pairs can be validated experimentally by uptake assays or spectroscopic characterization. The shell thickness, crosslink density, and surface chemistry can be tuned to balance binding kinetics, selectivity, and anti-fouling performance in the intended medium.

In certain embodiments, the core-shell nanoparticles may be formulated into printable inks or dispersions. Solvent systems comprising polar protic and aprotic components (for example, ethanol, water, and N-methylpyrrolidone) can be used to achieve stable colloidal dispersions and to tailor viscosity and surface tension for droplet-based deposition. Exemplary solvent ratios may include ethanol: water: N-methylpyrrolidone at 2:2:1 or 1:1:2 (v/v), with the choice informed by solvent polarity and particle-solvent interactions. Dispersions may contain, for example, about 5 mg mL-1 of core-shell nanoparticles and can be filtered to remove aggregates. When deposited, the nanoparticles can form uniform, densely packed layers on conductive or dielectric substrates while preserving the functional integrity of the MIP shell and the redox activity of the core. These embodiments may facilitate large-area, multiplexed integration in which different nanoparticle populations-each imprinted for a distinct analyte—are co-deposited or patterned as separate regions.

Embodiments can exhibit operational attributes for use in biofluid environments and for scalable manufacturing. The redox-active core can provide sustained electrochemical stability during extended cycling in buffer and biological matrices, and the MIP shell can confer selectivity and resistance to nonspecific fouling. The nanoparticles can be regenerated in situ by electrochemical or solvent-based procedures to remove bound targets and restore baseline response. The materials can be stored at ambient conditions for extended periods with retention of analytical performance and can maintain functionality under thermal excursions that would denature protein-based receptors. These features collectively may address recognized needs for durable, selective, and manufacturable recognition/transduction materials suited to continuous operation in complex media.

The present disclosure further contemplates methods of making the nanoparticles. In representative methods, a redox-active core comprising nanocubes may be synthesized by reacting a metal salt (e.g., nickel(II) acetate) with potassium hexacyanoferrate(III) in the presence of a chelating agent (e.g., trisodium citrate) under aqueous conditions, followed by isolation and washing. The cores may then be contacted with a solution containing a functional monomer (e.g., methacrylic acid), a crosslinker (e.g., ethylene glycol dimethacrylate), a template molecule (e.g., ascorbic acid, tryptophan, creatinine, cyclophosphamide, busulfan, mycophenolic acid), and an initiator (e.g., AIBN), and polymerized under controlled temperature and nitrogen atmosphere to form a conformal MIP shell. The template may be extracted to yield target-selective binding cavities, and the particles may be washed and dried. Variations may include alternative core compositions and morphologies, chelating agents, functional monomers, crosslinkers, initiators, polymerization temperatures and times, extraction solvents, and post-processing steps such as centrifugation, filtration, and vacuum drying.

Exemplary uses of the nanoparticles may include their incorporation into electrochemical sensing platforms configured for analysis of analytes in sweat, serum, or interstitial fluid. In such implementations, target recognition within the MIP shell of the deposited nanoparticles modulates the redox response of the confined core, enabling quantification through techniques such as differential pulse voltammetry. Multiplexed arrays can be formed by patterning distinct MIP/core populations, each selective for a different analyte class (e.g., vitamins, amino acids, metabolites, pharmaceuticals, etc.). These implementations may illustrate the versatility of the nanoparticles for continuous, selective monitoring in wearable or implantable formats, and are provided as non-limiting examples of the broader applicability of the disclosed core-shell nanoparticle compositions and methods.

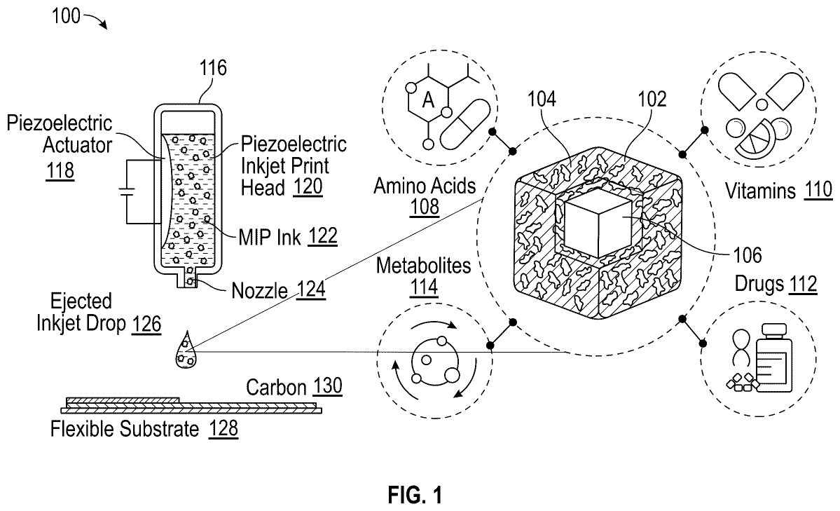

Turning now to the figures, FIG. 1 depicts an exemplary system of printable core-shell nanoparticles 100 configured for selective molecular recognition and electrochemical signal transduction, and for deposition via additive manufacturing. The system 100 may include at least one printable core-shell nanoparticle 102 comprising a shell 104 disposed conformally over a redox-active core 106. The shell 104 may be a molecularly imprinted polymer layer that presents target-specific binding cavities created by templating with representative small molecules. In operation, selective binding within the shell 104 modulates ion and electron transfer between the surrounding medium and the redox-active core 106, producing a measurable change in the core's redox response suitable for quantitative analysis.

The core 106 may provide the built-in electrochemical mediator for robust signal generation. In illustrative embodiments, the core 106 may comprise nickel hexacyanoferrate nanocubes (a Prussian blue analogue) selected for its crystallographic and redox stability during prolonged cycling in physiological media. The cubic morphology may offer high surface area and uniform pathways for alkali-ion intercalation, enabling stable and reversible redox peaks under techniques such as differential pulse voltammetry. Alternative cores can include other Prussian blue analogues (e.g., cobalt, copper, iron, manganese, zinc, vanadium, or chromium hexacyanoferrates), redox-active organic or organometallic moieties immobilized on or within nanoparticles (e.g., methylene blue, ferrocene derivatives, Azure dyes, toluidine blue O), and inorganic coordination complexes (e.g., ruthenium or osmium ammine complexes, cobaltocene). The composition, size, and shape of the core 106 may be tuned to adjust redox potential, kinetics, durability, and compatibility with the intended biofluid and readout modality.

The shell 104 may encode molecular selectivity via templated copolymerization around the core surface followed by template extraction to create cavities complementary in size, shape, and interaction motifs to the target analyte. Representative template categories may include but are not limited to amino acids 108, vitamins 110, drugs 112, and metabolites 114. By way of example, amino acid targets may include tryptophan or phenylalanine; vitamins may include ascorbic acid; drugs may include cyclophosphamide, busulfan, or mycophenolic acid; and metabolites may include creatinine, glucose, uric acid, or lactate. The shell 104 can be formed from functional monomers such as methacrylic acid and crosslinkers such as ethylene glycol dimethacrylate, initiated thermally or photochemically; alternative monomers (e.g., acrylamide, 4-vinylbenzoic acid, acrylic or itaconic acid, 2-vinylpyridine, N-vinylpyrrolidone, boronic acid-containing monomers, phosphorylcholine methacrylate, N-isopropylacrylamide) and crosslinkers (e.g., divinylbenzene, trimethylolpropane trimethacrylate, poly(ethylene glycol) diacrylate, N,N′-methylenebisacrylamide, sol-gel crosslinkers) can be used to tailor affinity, selectivity, permeability, anti-fouling behavior, and mechanical integrity. Shell thickness and crosslink density can be adjusted to balance binding kinetics and transport resistance.

The system 100 may further encompass deposition hardware and materials enabling scalable integration of the nanoparticles 102 into devices. An inkjet printer 116 may be used to deposit a dispersion of the nanoparticles in an MIP ink 122 with rheology tailored for drop-on-demand printing. A piezoelectric actuator 118 may drive volumetric deformation within a piezoelectric inkjet printhead 120 to generate and expel discrete volumes of the MIP ink 122 through a nozzle 124 as an ejected inkjet drop 126. The ejected inkjet drop 126 may land on a flexible substrate 128 in programmed patterns, forming uniform and densely packed nanoparticle films that can retain the functional shell 104 and the redox activity of the core 106. Alternative actuation mechanisms for droplet generation can include thermal bubble, electrostatic, or acoustic methods; alternative print technologies can include aerosol jet, screen printing, microdispensing, and spray or spin coating for different resolution, throughput, and material compatibility requirements. Nozzle 124 geometry and operating waveforms can be tuned to manage drop volume, jetting stability, and feature definition.

The MIP ink 122 may comprise the printable medium containing the nanoparticles 102, typically dispersed in a mixed solvent system that balances polarity, volatility, viscosity, and surface tension to promote stable jetting and uniform film formation. Solvent blends may include combinations of protic and aprotic solvents, for example water, ethanol, and N-methylpyrrolidone, with ratios selected to minimize particle aggregation. Dispersants, humectants, or surfactants can be included to control drying dynamics and line edge quality, provided they do not compromise shell 104 integrity or core 106 redox behavior. Alternative formulations can employ other alcohols, glycols, lactams, or green solvents compatible with bioelectronics fabrication and device substrates.

The flexible substrate 128 may provide a mechanically compliant platform suitable for wearable or implantable applications and can support patterned conductors, insulators, and encapsulants. Suitable materials include polymer films such as polyethylene terephthalate, polyimide, thermoplastic polyurethane, and medical-grade elastomers. Conductive features on the flexible substrate 128 can include carbon 130 as a printed working electrode or underlayer that enhances electron transfer and provides a compatible surface for nanoparticle adhesion. Alternatives for the conductive layer include printed gold, silver/silver-chloride, platinum, graphene, carbon nanotube composites, and conductive polymers such as PEDOT:PSS. Additional dielectric or passivation layers can be added to define microfluidic pathways, isolate interconnects, and protect electronics while leaving sensing areas exposed to the target biofluid.

In use, patterned regions containing different populations of nanoparticles 102 (each imprinted for a distinct target class such as amino acids 108, vitamins 110, drugs 112, or metabolites 114) can be co-fabricated on the same flexible substrate 128 to form multiplexed arrays. During operation in a biofluid, targets bind into the shell 104 cavities, partially occluding ion pathways and altering interfacial charge transfer to the core 106. This may manifest as changes in electrochemical signatures (e.g., decreased peak current in voltammetry, increased charge-transfer resistance in impedance spectroscopy) that correlate with analyte concentration. The confined mediator architecture may minimize leaching and support repeated use; bound targets can be removed by applying a regeneration potential or rinsing in an appropriate solvent, restoring baseline response for continuous monitoring.

The components of the system 100 may be designed to work in concert to enable scalable, high-performance molecular sensing. The inkjet printer 116 and printhead 120 may deliver the MIP ink 122 with high placement accuracy to define sensing pixels and interconnects; the nozzle 124 and ejected inkjet drop 126 may govern feature size and uniformity; the flexible substrate 128 and carbon 130 layers may provide mechanical support and electrical pathways; and the printed nanoparticles 102 may furnish both recognition via the shell 104 and transduction via the core 106. By selecting appropriate template families 108, 110, 112, 114 and by tuning materials and printing parameters, the same platform can be adapted to a wide range of targets and device formats, including wearable sweat patches, microneedle-based interstitial fluid sensors, and subcutaneous implantable arrays.

While FIG. 1 illustrates a representative piezoelectric inkjet configuration and carbon-based electrode architecture, the system 100 is not limited to these implementations. For example, the core 106 can be engineered as nanospheres or nanoframes rather than nanocubes; the shell 104 can incorporate zwitterionic or PEGylated motifs to enhance anti-fouling; the MIP ink 122 can be optimized for alternative nozzles or substrates; and the flexible substrate 128 can be replaced with rigid glass, silicon, or ceramic carriers for benchtop or in vitro diagnostic devices. Such variations can be implemented without departing from the discussed concept of a printable, molecule-selective core-shell nanoparticle platform for robust electrochemical sensing.

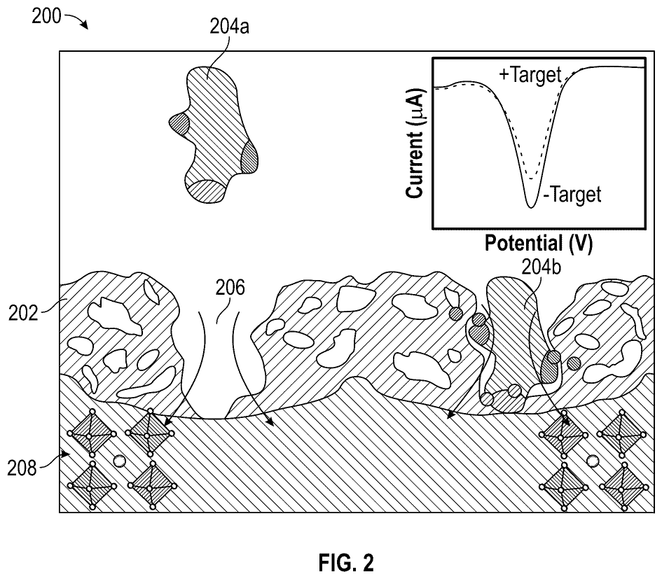

FIG. 2 illustrates an example electrochemical sensing mechanism 200 implemented by a core-shell nanoparticle architecture in which an MIP 202 may be disposed over a redox-active core 208. In operation, the MIP 202 may confer molecular recognition by presenting shape-and chemistry-complementary sites to selected analytes, while the core 208 may furnish a stable, reversible redox transducer comprised within the nanoparticle. When the nanoparticle is immersed in a biofluid or buffer and interrogated electrochemically, changes in access of ions and electrons to the core 208 (regulated by target binding within the MIP 202) may be transduced into measurable signal changes (for example, in differential pulse voltammetry or electrochemical impedance spectroscopy).

The figure further depicts a portion of the imprinting process that creates the recognition features in the MIP 202. During polymerization, a non-extracted template molecule 204b may be present and associate with functional monomer(s) at or near the surface of the core 208 through noncovalent or reversible covalent interactions (e.g., hydrogen bonding, ionic pairing, π-π interactions, or boronate-diol complexation). Polymer growth and crosslinking may fix the three-dimensional arrangement of these interactions around the non-extracted template molecule 204b. After polymerization, removal of the template (represented as an extracted template molecule 204a) may leave behind target-selective binding cavities 206 distributed through the MIP 202 shell. Each target-selective binding cavity 206 may be complementary to the template in size, shape, and functional group orientation, enabling selective rebinding of the intended analyte during sensing.

In a “background” state before analyte binding, the MIP 202 may be porous enough to permit solvated ions to reach the redox-active sites of the core 208, supporting rapid charge transfer and yielding a characteristic redox peak height and position. Upon exposure to a sample containing the target analyte, occupancy of the target-selective binding cavities 206 by the analyte increases local tortuosity and partially occludes ion transport pathways at the core-electrolyte interface. This binding-induced modulation may manifest as reduced peak currents in voltammetric readouts and/or increased charge-transfer resistance in impedance measurements, with the magnitude of the change correlating with analyte concentration over a defined dynamic range. Shell thickness, crosslink density, and monomer chemistry can be tuned to balance binding affinity and transport, thereby controlling sensitivity, selectivity, and response time.

The core 208 may operate as an embedded redox mediator whose signal may be coupled to the interfacial conditions imposed by the MIP 202. In some embodiments, the core 208 may comprise a Prussian blue analogue (e.g., nickel hexacyanoferrate) that exhibits highly reversible alkali-ion intercalation/deintercalation with minimal lattice strain, allowing cycling stability in physiologic media. However, the mechanism 200 may be agnostic to the precise core chemistry and can be implemented with other stable redox-active materials provided they deliver a clear, reproducible signal within the potential window compatible with the target environment.

FIG. 2 also highlights the reversible nature of sensing. After a measurement, bound target within the target-selective binding cavities 206 can be released by applying a suitable regeneration potential or by brief exposure to a benign extraction solvent, thereby resetting the MIP 202 to its background state without degrading the core 208. This regeneration capability enables continuous or repeated measurements with minimal drift, extending operational lifetime and reducing consumable needs compared to systems that rely on soluble mediators or fragile bioreceptors.

Alternative embodiments of the electrochemical sensing mechanism 200 can employ different interaction chemistries within the MIP 202 to accommodate diverse targets, including acids, bases, neutral aromatics, saccharides, vitamins, amino acids, and therapeutic drugs. The role of the non-extracted template molecule 204b and extracted template molecule 204a remains similar across these variants: the former may define the preorganized monomer-template complex during imprinting, and the latter may represent the removed species that gives rise to the functional target-selective binding cavities 206. Likewise, while the depiction focuses on voltammetric transduction at the core 208, the same or similar binding-controlled transport modulation can be read out by other electroanalytical techniques (e.g., square-wave voltammetry, chronoamperometry, or impedance), depending on device design and application constraints.

Finally, the mechanism 200 may support multiplexing by patterning distinct nanoparticle populations, each with its own MIP 202 and corresponding target-selective binding cavities 206, onto a shared electrode platform. In such arrays, each population may respond independently to its cognate analyte through the same binding-transport-transduction pathway centered on the respective core 208, enabling simultaneous, selective quantification of multiple biomarkers in complex biofluids.

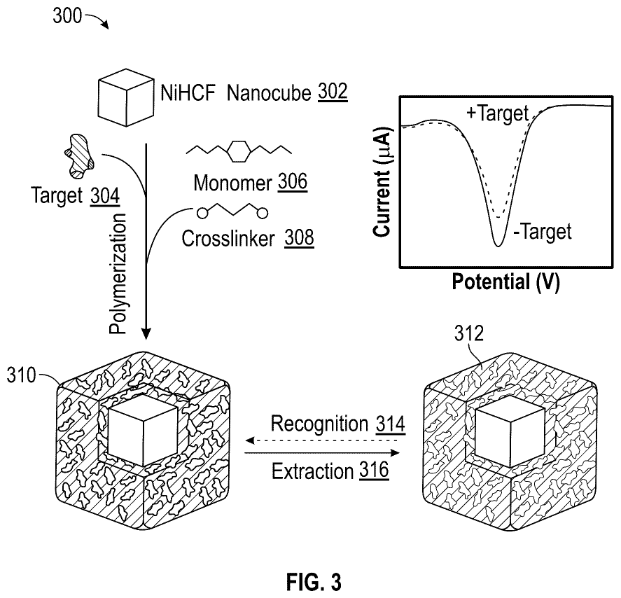

FIG. 3 depicts an example solution synthesis of a printable core-shell nanoparticle 300 that integrates a redox-active nickel hexacyanoferrate core with a molecularly imprinted polymer shell. The process may begin with preparation of a NiHCF nanocube 302, which may function as a mechanically and electrochemically stable scaffold. In various embodiments, the nanocube may be the core (e.g., core 106, 208) The nanocube may offer uniform cubic morphology and robust, reversible redox behavior in physiologic media, making it a relevant transducer around which a selective polymer shell can be constructed. The aqueous synthesis can be chelate-modulated to yield monodisperse particles with clean surfaces that support subsequent interfacial polymerization.

The selective shell may be defined by introducing a target molecule 304 together with a monomer 306 and a crosslinker 308 in a solvent system that disperses and wets the core surface. The target molecule may associate with the functional monomer via noncovalent or reversible covalent interactions, pre-organizing a monomer-template complex at the nanocube interface. Upon initiation, copolymerization and crosslinking may proceed at and around the surface of the NiHCF nanocube 302, thereby fixing the spatial arrangement of functional groups around the target. This stage may yield a nanocube with a template molecule 310, in which the polymer network conforms to the molecular contours and interaction motifs presented by the target molecule.

Following shell formation, an extraction 316 step may remove the templating species from the polymer network without degrading the underlying redox core or the integrity of the shell. After the template is removed, the product may be a nanocube with a template molecule extracted 312, which may comprise a population of target-complementary voids (e.g., target-selective binding cavities) distributed throughout the shell. These cavities may be defined by the steric shape and the chemical environment set during polymerization, and they may preserve accessibility for rebinding in subsequent analytical use.

In operation, recognition 314 may occur when the target molecule 304 in a sample rebinds into the target-selective binding cavities of the imprinted shell. This occupancy modulates ionic and electronic transport at the shell-electrolyte-core interface, altering access of charge carriers to the NiHCF nanocube 302 and producing quantifiable changes in electrochemical readouts. The balance of monomer 306 and crosslinker 308, as well as polymerization and extraction 316 conditions, can be tuned to control shell thickness, crosslink density, permeability, and affinity, thereby tailoring sensitivity, selectivity, and response time. The resulting core-shell nanoparticles can be dispersed into stable inks for digital deposition, enabling scalable fabrication of multiplexed sensing elements while maintaining the recognition 314 capability encoded during the solution synthesis of a printable core-shell nanoparticle 300.

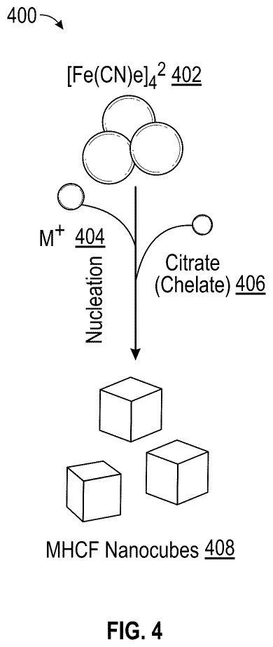

FIG. 4 illustrates an exemplary chelate-assisted synthesis of Prussian blue analogues as the core of a printable core-shell nanoparticle 400. In this approach, a hexacyanoferrate precursor [Fe(CN)e]₄² 402 may be reacted with a transition-metal cation source M+404 under rate-controlled conditions established by a chelating agent such as citrate 406. Citrate 406 can transiently complex the metal ion M+ 404, moderating the instantaneous free-ion activity and thereby slowing the coordination reaction with the cyanoferrate ligand 402. This controlled release of M+ 404 may favor uniform nucleation events followed by facet-selective crystal growth, yielding well-defined metal-hexacyanoferrate frameworks as monodisperse MHCF nanocubes 408 suitable for use as redox-active cores.

As depicted, the hexacyanoferrate species 402 may provide the octahedral [Fe-CN] building blocks that bridge with the chelated metal M+ 404 once partially liberated from the citrate 406 complex. By tuning chelation strength, pH, ionic strength, and addition rate, the supersaturation profile can be shaped to decouple nucleation from growth, promoting the cubic morphology and narrow size distributions of the MHCF nanocubes 408. Representative metals for M+ 404 may include Ni, Co, Cu, and Fe, enabling formation of NiHCF, CoHCF, CuHCF, and FeHCF, respectively; among these, nickel hexacyanoferrate may be selected for its zero/low-strain ion intercalation and cycling stability in physiological media. Process variants may substitute or supplement citrate 406 with other chelators (e.g., EDTA, oxalate, tartaric acid, triethanolamine) and may employ controlled injection, temperature regulation, and aging to further refine crystal quality.

The resulting MHCF nanocubes 408 may be isolated by centrifugation and washing to remove excess chelate 406 and unreacted precursors, affording cleaner, dispersible crystals that retain higher crystallinity and electrochemical activity. These nanocubes may provide a high-surface-area, redox-stable scaffold that interfaces with subsequently formed molecularly imprinted shells, enabling the printable core-shell architectures described herein.

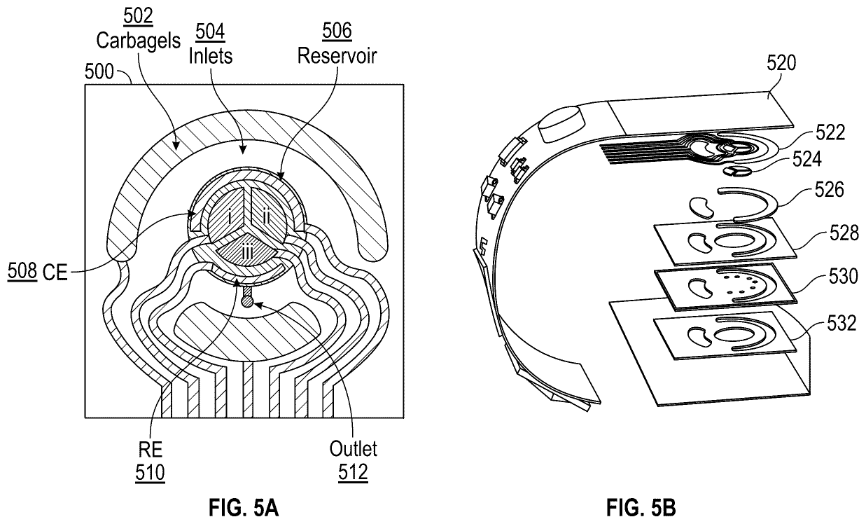

FIG. 5A illustrates an example flexible and wireless microfluidic wearable patch 500 that provides an example platform for implementing the printable core-shell nanoparticles described herein. The patch 500 may incorporate carbagel 502, which may be a carbachol-loaded hydrogel positioned to deliver iontophoretic stimulation that induces sweat locally and on demand. Iontophoresis-driven sweat may be routed into a network of inlets 504 that feed a patterned reservoir 506. The reservoir 506 may serve as a transient sampling volume for electrochemical analysis and may be fluidically coupled to an outlet 512 that continuously evacuates spent sample, thereby enabling time-resolved measurements with minimal carryover and mitigating evaporation or analyte degradation. The microfluidic topology can be defined in elastomeric or adhesive laminates laminated onto a flexible substrate, and the overall construction may be conformal to the skin to maintain a stable interface during daily activity.

Electrochemical transduction within the patch 500 may be provided by an electrode set that includes a counter electrode 508 and a reference electrode 510 positioned to contact the sampled sweat within the reservoir 506. The printable core-shell nanoparticle sensing layers (such as molecularly imprinted polymer shells on nickel hexacyanoferrate cores) may be deposited as the active sensing interface on or near the counter electrode 508, with the reference electrode 510 furnishing a stable potential for differential pulse voltammetry, impedance spectroscopy, or related techniques. During operation, iontophoresis via the carbagel 502 may induce sweat that can be continuously delivered through the inlets 504, interrogated electrochemically in the reservoir 506 against the reference electrode 510, and removed via the outlet 512, enabling repeated sampling, regeneration, and multiplexing on a compact, low-power, wireless platform.

This example system demonstrates one suitable implementation in which the nanocube-based recognition/transduction layers can be integrated for noninvasive molecular monitoring. Variations may employ passive sweat collection instead of iontophoresis, alternative hydrogel formulations for the carbagel 502, different microfluidic geometries for the inlets 504 and reservoir 506, or alternative electrode materials and layouts while retaining the same functional roles of the counter electrode 508, reference electrode 510, and outlet 512 within a flexible, skin-interfaced, and wirelessly connected architecture. Further embodiments and implementations of the nanoparticles may exist.

For example, the nanoparticles can be implemented in a variety of device formats tailored to different use environments and biofluids. For example, subcutaneous implantable sensor arrays can be fabricated by printing the nanoparticle layers onto miniaturized flexible substrates and encapsulating them with biocompatible, porous membranes to enable continuous interstitial fluid monitoring with wired or wireless telemetry. Microneedle-based platforms can incorporate the nanoparticles on the needle shafts or tips to access interstitial fluid with minimal pain, supporting multiplexed drug and metabolite sensing with on-body regeneration cycles. For point-of-care testing, the particles can be deposited onto screen-printed electrodes or paper-based microfluidics to create low-cost, disposable cartridges that quantify targets in fingerstick blood, serum, saliva, or urine using handheld potentiostats. Catheter-, stent-, or implant-coatings that carry the nanoparticle films can provide local, real-time pharmacokinetic readouts or inflammation monitoring directly at indwelling device interfaces. In oral or ocular applications, the particles can be integrated into mouthguards or contact-lens-compatible electrode laminates to track salivary or tear biomarkers using thin, transparent conductors. The printable inks also enable high-density arrays on rigid substrates (e.g., glass, silicon, ceramics) for benchtop analyzers and automated microfluidic chips, allowing parallel assays with on-chip regeneration and minimal cross-talk; similarly, textile-integrated electrodes and watchband or ring inserts can host the nanoparticle films for passive or induced sweat analysis without discrete patches. Across these embodiments, the core-shell architecture may provide target recognition and stable electrochemical transduction, while fabrication can leverage inkjet, aerosol jet, screen printing, or microdispensing to conformally pattern the sensing layers onto the chosen form factor.

FIG. 5B illustrates an example layered design of a flexible and wireless microfluidic wearable patch that can implement the printable core-shell nanoparticles disclosed herein. In the depicted embodiment, a plastic substrate 520 may interface mechanically and electrically with a printed circuit board to support device interconnection, signal conditioning, and wireless telemetry. On the substrate 520, inkjet-printed electrodes and interconnects 522 may define working, counter, and reference electrode features and associated conductive routing. Over selected electrode sites, an inkjet-printed MIP/NiHCF nanoparticle sensing layer 524 may be patterned as the active recognition/transduction interface. The fluid-handling stack above the sensing region may include carbachol-loaded hydrogels (carbagels) 526 positioned to provide iontophoretic stimulation for on-demand sweat induction, a reservoir layer 528 may be configured to hold and refresh samples over the sensing area, and an inlet layer 530 may route induced sweat from collection points to the reservoir layer 528. A sweat accumulation layer 532 may be disposed to directly interface with the skin to collect locally generated sweat and to establish fluidic continuity to the inlets.

In operation, iontophoresis via the carbagels 526 may promote sweat generation at the skin-contacting sweat accumulation layer 532. The sweat may be directed through the inlet layer 530 into the reservoir layer 528, where it transiently contacts the inkjet-printed MIP/NiHCF nanoparticle sensing layer 524 over the electrodes 522 for electrochemical interrogation (e.g., differential pulse voltammetry). Target molecules may selectively bind within the molecularly imprinted polymer shell of the sensing layer 524, modulating interfacial transport to the redox-active NiHCF core and producing a concentration-dependent change in the recorded signal. Sample refresh through the reservoir layer 528 can reduce carryover, support repeated regeneration of the sensing layer 524, and enable multiplexed measurement when multiple imprinted nanoparticle populations are patterned on distinct electrode sites within the same stack.

The layer assignments in FIG. 5B provide one non-limiting embodiment that leverages the printable, core-shell nanoparticle architecture for skin-interfaced chemical sensing. Variations may include alternative substrate materials for the plastic substrate 520, different conductor and encapsulant chemistries for the electrodes and interconnects 522, alternative hydrogel formulations or passive collection in place of the carbagels 526, modified channel geometries in the inlet layer 530 and reservoir layer 528 to tune residence time and washout, and optional barrier or porous membranes adjacent the sensing layer 524 to enhance anti-fouling while preserving mass transport. In each case, the MIP/NiHCF nanoparticle sensing layer 524 may remain the active element providing target-selective molecular recognition and robust electrochemical transduction within a scalable, inkjet-printed wearable format.

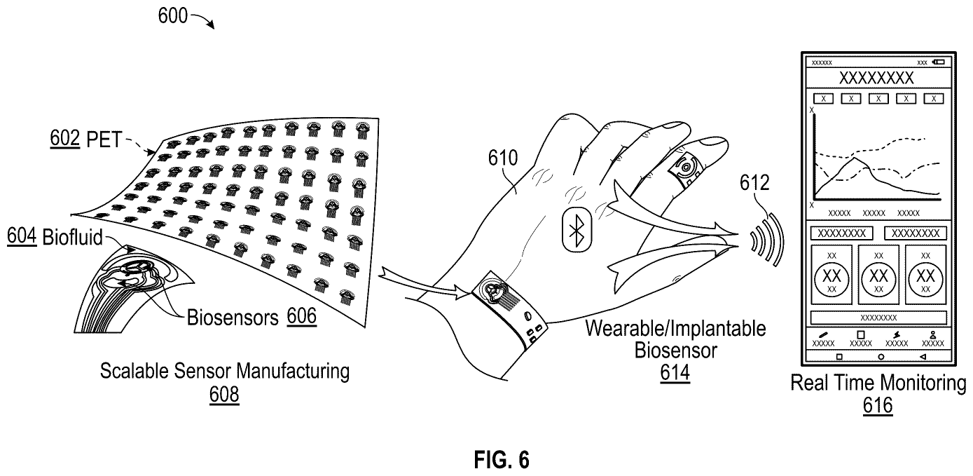

FIG. 6 illustrates an example of mass-produced flexible and wireless microfluidic wearable patches and an example implementation thereof 600. In this depiction, device layers may be patterned onto a flexible polymer substrate such as PET 602 using additive processes that enable scalable sensor manufacturing 608. Conductive traces, electrode sites, and encapsulation features may be serially deposited, and the active sensing regions may be defined by printing dispersions of the core-shell nanoparticles described herein as the recognition/transduction layer. The assembly may integrate low-profile electronics for power management and signal conditioning and include a compact Bluetooth module to provide wireless communication 612 to external devices for data streaming and control.

Within the device, arrays of biosensors 606 may be formed by patterning distinct nanoparticle populations (each carrying a molecularly imprinted polymer shell over a redox-active core) onto designated electrode sites. Microfluidic structures may guide a biofluid 604 (such as sweat collected on the skin surface or interstitial fluid accessed through minimally invasive interfaces) over the sensing areas while managing refresh and washout to support continuous operation. As shown in the example implementation, the patch may conform to and operates on a hand 610 (or other body part), with the flexible stack and adhesive layers maintaining intimate skin contact for measurements during motion. The nanoparticle-enabled sensing pixels transduce target binding into electrochemical signals that are acquired, processed, and transmitted wirelessly for downstream visualization and analysis.

The printed sensing architecture can be configured as a wearable/implantable biosensor 614, enabling in situ measurements on the body surface or subcutaneously. For implantable formats, the nanoparticle-coated electrodes may be laminated with biocompatible, porous overlays to moderate tissue interaction while preserving mass transport to the active layer. In both wearable and implantable embodiments, the nanoparticle films may provide selective recognition and stable redox transduction for real-time monitoring 616 of diverse analytes, including vitamins, amino acids, metabolites, and therapeutic drugs. Regeneration steps (such as brief electrochemical potentials) can be applied between measurement cycles to clear bound targets and sustain performance over extended use.

Production of these devices can leverage scalable sensor manufacturing 608 pathways, including inkjet or roll-to-roll deposition on PET 602 and related flexible substrates, to produce large volumes of uniform sensor arrays at low cost. The integrated Bluetooth module may enable wireless communication 612 with mobile or bedside systems for configuration, time stamping, and secure data transfer, supporting longitudinal, cloud-connected real-time monitoring 616 in research or clinical environments. While PET 602 is highlighted for its printability and mechanical robustness, other flexible films (e.g., polyimide or thermoplastic polyurethane) may be substituted, and the biosensors 606 may be adapted to alternative form factors (such as bands, rings, patches, or implantable strips) without departing from the nanoparticle-based sensing implementation discussed herein.

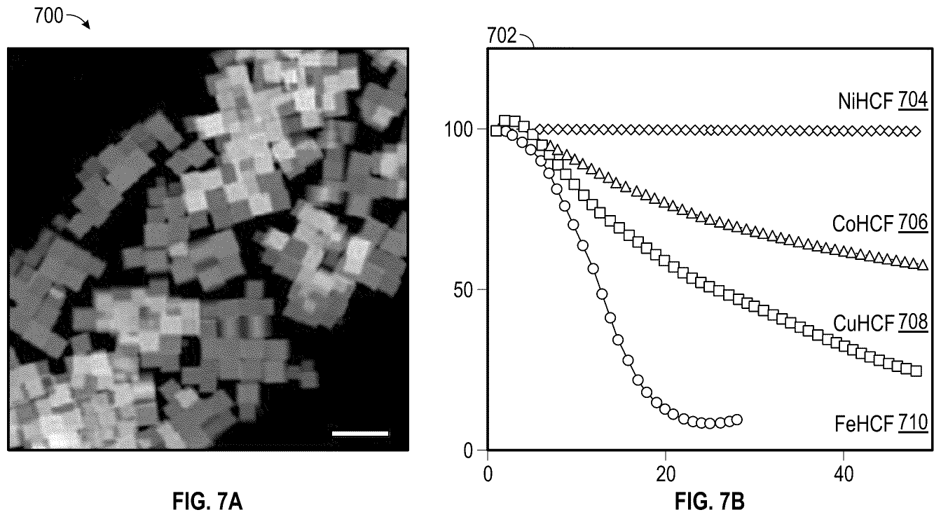

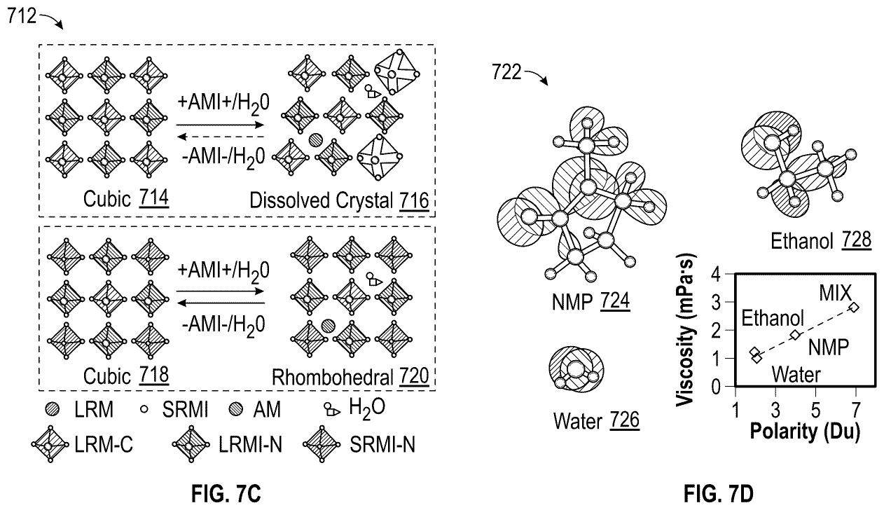

FIG. 7A presents exemplary imaging of printable core-shell nanoparticles 700, including transmission electron microscopy and dark-field scanning transmission electron microscopy that resolve uniform nickel hexacyanoferrate nanocubes (approximately 100 nm edge length) conformally coated with a thin molecularly imprinted polymer shell visible as a lower-contrast rim. Elemental mapping by energy-dispersive X-ray spectroscopy can confirm homogeneous nickel and iron distribution within the crystalline core and oxygen-rich signal consistent with the polymeric shell at the particle periphery, while high-resolution images can indicate preserved lattice fringes indicative of retained crystallinity after shell formation. Complementary scanning electron microscopy of inkjet-printed films can reveal densely packed, continuous nanoparticle layers on conductive substrates, and cross-sectional views can demonstrate controlled stack thickness and uniform coverage suitable for multiplexed electrode patterning. Collectively, these imaging modalities can verify the intended core-shell architecture, shell conformity and thickness, elemental localization, and print-induced film morphology that underpin selective molecular recognition and robust electrochemical transduction in device implementations.

FIG. 7B presents exemplary cyclic voltammogram oxidation peak current changes of various materials for printable core-shell nanoparticles 702, comparing NiHCF 704, CoHCF 706, CuHCF 708, and FeHCF 710 under repetitive electrochemical cycling in physiologically relevant media. The traces demonstrate that NiHCF 704 maintains the highest redox stability with minimal decay in oxidation peak current over thousands of cycles, consistent with zero/low-strain alkali-ion intercalation in its robust lattice; CoHCF 706 shows moderate stability with some attenuation of peak amplitude; CuHCF 708 exhibits more pronounced degradation; and FeHCF 710 displays the most rapid loss of signal and structural integrity under identical conditions. This comparative performance underscores the suitability of NiHCF 704 as a relevant redox-active core for long-term wearable and implantable sensing, while highlighting that alternative Prussian blue analogues can be employed when different redox potentials or cost constraints are desired, with an informed trade-off in cycling durability.

FIG. 7C illustrates an exemplary ion intercalation mechanism for printable core-shell nanoparticles 712, emphasizing how alkali-ion insertion and extraction influence the crystal lattice of Prussian blue analogue cores during electrochemical cycling. In a zero/low-strain pathway characteristic of nickel hexacyanoferrate, the framework remains cubic 714 during ion uptake and retains a stable cubic 718 configuration upon release, supporting reversible Na+/K+ transport and reproducible redox signaling. By contrast, compositions with less favorable lattice accommodation can undergo symmetry changes to a rhombohedral 720 phase under repeated intercalation, increasing mechanical stress, impeding ion transport, and accelerating performance decay. In still less stable scenarios, prolonged cycling in physiological electrolytes can promote lattice breakdown and species loss, depicted as a dissolved crystal 716, which diminishes redox activity and structural integrity. This comparative behavior underscores the use of zero/low-strain cubic frameworks for durable, high-fidelity transduction in the core of the printed core-shell architecture.

FIG. 7D presents an example of highest occupied molecular orbitals of solvent molecules used to disperse printable core-shell nanoparticles 722, illustrating how orbital distributions correlate with solvent polarity and dipole-driven interactions that stabilize nanoparticle inks. The depiction compares NMP 724 (N-methylpyrrolidone), water 726, and ethanol 728, where the extended, highly polarized HOMO character of NMP 724 and the strong polarity of water 726 favor robust dipole-dipole and ion-dipole interactions with the molecularly imprinted, redox-active particles, thereby suppressing self-aggregation and promoting colloidal stability. Ethanol 728, while less polar than water 726, contributes favorable wetting, volatility, and surface-tension balancing for drop-on-demand jetting. In mixed-solvent formulations (e.g., NMP 724 with water 726 and ethanol 728), the combined HOMO-informed polarity profile helps shield particle-particle interactions, mitigate ring effects, and yield uniform, densely packed printed films that preserve shell integrity and core electroactivity.

Additional embodiments, characterizations, and implementation details.

Electrochemical operation and readout. The disclosed nanoparticles may be readily interrogated by differential pulse voltammetry, cyclic voltammetry, amperometry, and impedance spectroscopy to translate target binding into measurable signal changes. Representative DPV settings for biofluid analysis may include a potential window from 0.8 to 0 V, 10 mV step, 50 mV pulse amplitude, 50 ms pulse width, 0.5 s period, and 1×10−5 A V−1 sensitivity, for example. Open-circuit potential-EIS can be used to track construction and operation, with frequency sweeps from 0.1-10\hat{ }6 Hz and 5 mV excitation; printing the nanoparticle layer may reduce impedance versus the bare carbon substrate, while target binding may increase charge-transfer resistance and total impedance. In one implementation, each on-body measurement cycle may begin with an electrochemical cleaning/conditioning step, followed by a “background” DPV scan without incubation, a controlled incubation period, and a post-incubation DPV scan; a brief regeneration potential may then be applied to remove bound targets and restore baseline.

Ink formulation and printing process. To achieve stable drop-on-demand jetting and uniform films, core-shell nanoparticles may be dispersed as an ink in mixed solvent systems in which polarity and volatility are tuned computationally and empirically. Solvents with higher dipole moments and favorable HOMO distributions (e.g., N-methylpyrrolidone and water) may suppress particle self-interaction and aggregation, while lower-surface-tension cosolvents (e.g., ethanol) may improve wetting and drying. Illustrative blends may include ethanol:water:NMP at 2:2:1 or 1:1:2 (v/v). Inks at approximately 5 mg mL-1 solids are ultrasonically dispersed, filtered (e.g., 0.45 μm), printed onto oxygen plasma-treated polymer films using heated platens (e.g., 50° C.) to promote rapid solvent removal, and subsequently annealed (e.g., 90° C.) to stabilize film morphology.

Scalable additive manufacturing and layer stack. The devices may be produced by serially printing conductive and sensing layers onto flexible films (e.g., PET), including gold interconnects, carbon working/counter electrodes, silver reference electrodes, encapsulation (e.g., SU-8), and the core-shell nanoparticle films. Cross-sectional and plan-view electron microscopy of printed electrodes can confirm dense packing of nanocubes atop the carbon underlayer and uniform coverage. Performance can be optimized by adjusting the number of nanoparticle passes; in one embodiment, four printed layers yield a film thickness of about 200 nm and a roughness of about 200 nm with superior analytical response. Mass-production feasibility may be evidenced by electrochemical characterization of more than one thousand printed sensors, which show tight distributions of redox peak currents and consistent calibration across independent fabrication batches.

Durability, anti-fouling, regeneration, and thermal resilience. The core-shell nanoparticles may maintain function during extended exposure to physiologic media and mechanical stressors. In PBS, sweat, and serum, repeated voltammetric scans (e.g., 100-500 cycles) can show minimal drift and preserved redox features, indicating anti-biofouling behavior. Printed devices can retain over 80% of analytical sensitivity after four months of room-temperature storage. Electrochemical regeneration may restore signal repeatedly by brief potential holds (e.g., −0.3 V for 60 s for an ascorbic acid sensor), enabling long-term, continuous operation. The MIP-based architecture may exhibit superior thermal robustness relative to protein-or nucleic acid-based receptors; for example, glucose sensors fabricated with the disclosed nanoparticles may retain a substantial fraction of response after 2 hours at 90° C., whereas enzymatic comparators lose most of their signal under the same treatment. Flexural endurance may be demonstrated by stable sensor response over 1,200 bending cycles at radii consistent with wearable use.

Microfluidics, iontophoresis, and wearable electronics. Wearable patches may integrate multi-inlet microfluidic sampling layers with a reservoir and outlet to continuously refresh samples over the sensing area, thereby minimizing carryover and evaporation effects. On-demand sweat induction at rest may be achieved via iontophoresis using carbachol-loaded agarose hydrogels (“carbagel(s)”) paired with NaCl gels at the counter-electrode; representative formulations include 3% w/w agarose with 1% w/w carbachol (anode) and 1% w/w NaCl (cathode). Flexible electronics may provide power management, analog front-end control for multi-channel electrochemical measurements (e.g., DPV), and Bluetooth Low Energy (BLE or Bluetooth) telemetry for real-time data streaming to a mobile app or computing system. A compact, finger-or wrist-worn form factor enables autonomous cycles of cleaning, background scan, incubation, readout, and regeneration.

Human studies and correlations between sweat and serum. For metabolic and nutritional monitoring, the wearable system may quantify selected biomarkers in sweat and demonstrate strong correlations with serum levels; representative Pearson coefficients for ascorbic acid, creatinine, and tryptophan are approximately 0.87, 0.81, and 0.86, respectively. On-body measurements distinguish cohorts, with individuals reporting long COVID symptoms exhibiting lower ascorbic acid and tryptophan and higher creatinine compared to healthy controls, consistent with reported blood trends. Nutritional challenges—such as consumption of a protein shake or targeted supplements—produce rapid increases in sweat ascorbic acid and tryptophan across participants, while creatinine remains relatively stable over the same short time horizons, supporting physiological plausibility of the signals and responsiveness of the system.

Therapeutic drug monitoring and clinical validation. Drug-imprinted nanoparticles for cyclophosphamide, busulfan, and mycophenolic acid may show log-linear calibration by DPV. In hospitalized cancer patients, sweat drug concentrations measured by the wearable sensors correlate with LC-MS/MS results, with correlation coefficients up to approximately 0.98, indicating analytical validity. Time courses of sweat drug levels recorded on-body mirror expected plasma pharmacokinetics after infusion. For preclinical pharmacokinetics, subcutaneously implanted, multiplexed arrays in mice capture dose-dependent exposure (e.g., area-under-the-curve changes with cyclophosphamide dose) and resolve sequential administration of multiple immunosuppressants with selective responses.

Computational design and materials selection. Computational workflows may inform both shell chemistry and ink engineering. Monomer selection for imprinting may be guided by docking and density functional theory to maximize binding energy to the target and binding energy differentials against likely interferents; for ascorbic acid, methacrylic acid provides an optimal balance of predicted sensitivity and selectivity, confirmable experimentally by higher uptake and spectroscopic signatures. Solvent choices may be screened by ab initio calculations of dipole moments and frontier orbitals to favor strong dipole-particle interactions and stable dispersion. Separate first-principles analysis of Prussian blue analogues elucidates lattice energetics, supporting the selection of nickel hexacyanoferrate cores with zero/low-strain ion intercalation pathways that preserve the cubic framework during repeated cycling in physiologic electrolytes.

Cost, scalability, and manufacturing considerations. The additive manufacturing route supports low per-sensor material costs and high throughput. An illustrative bill of materials for a multiplexed patch indicates sub-dollar per-sensor costs for inks and substrate (e.g., carbon, gold, silver, encapsulant, nanoparticle ink, and PET), with the nanoparticle layer deposited in a single printing step per channel. Laser-patterned adhesives and polymer laminates may define the microfluidic stack at scale, and the printing workflow may be compatible with roll-to-roll or batch panel processing. Device-to-device consistency may be supported by eliminating manual receptor modification and relying on digitally patterned, pre-functionalized nanoparticles with integrated transduction.

The disclosed technology provides a unified, printable core-shell nanoparticle platform that integrates selective molecular recognition and robust electrochemical transduction into a single, mass-producible material. A redox-stable Prussian blue analogue core may deliver long-lived, reversible signals in biofluids, while a molecularly imprinted polymer shell may encode analyte specificity and resist fouling. Computational design may streamline monomer and solvent selection, and inkjet-compatible formulations may enable precise, large-area deposition onto flexible substrates to build multiplexed wearable and implantable devices. The sensors may demonstrate reproducible manufacturing, room-temperature shelf stability, repeated electrochemical regeneration, mechanical and thermal resilience, cytocompatibility, and in vivo biocompatibility. The platform may support continuous, real-time measurement with close agreement to gold-standard assays. By unifying recognition and transduction at the nanoparticle level and aligning materials and processes with scalable printing, disclosed approaches may address longstanding barriers to broad deployment of molecular biosensing and position the technology for translation across clinical, research, and consumer health applications.

It should be understood that the various features, aspects and functionality described in one or more of the individual embodiments are not limited in their applicability to the particular embodiment with which they are described. Instead, they can be applied, alone or in various combinations, to one or more other embodiments, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like; and adjectives such as “conventional,” “traditional,” “normal,” “standard,” “known.” Terms of similar meaning should not be construed as limiting the item described to a given time period or to an item available as of a given time. Instead, they should be read to encompass conventional, traditional, normal, or standard technologies that may be available or known now or at any time in the future. Where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

The terms “approximately” and “substantially” are used to account for variations that may occur due to manufacturing tolerances, measurement inaccuracies, and other practical considerations in implementing the described technology. The term “approximately” refers to a value or range that is close to the stated value but allows for minor deviations, typically within ±10%, unless otherwise specified, that do not materially affect the function or purpose of the invention. Similarly, the term “substantially” is used to indicate that a particular feature, characteristic, or result is largely present or achieved, with allowable variations without deviating from the intended scope and function of the invention. These terms should be interpreted in a manner consistent with the understanding of a person skilled in the art.

It should be noted that the terms “optimize,” “optimal” and the like as used herein can be used to mean making or achieving performance as effective or perfect as possible. However, as one of ordinary skill in the art reading this document will recognize, perfection cannot always be achieved. Accordingly, these terms can also encompass making or achieving performance as good or effective as possible or practical under the given circumstances or making or achieving performance better than that which can be achieved with other settings or parameters.

Additionally, the various embodiments set forth herein are described in terms of exemplary block diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A nanoparticle, comprising:
a redox-active core; and
a shell disposed on an outer surface of the redox-active core, the shell comprising a plurality of target-specific binding cavities.

2. The nanoparticle of claim 1, wherein the shell is a molecularly imprinted polymer.

3. The nanoparticle of claim 1, wherein the redox-active core comprises a Prussian blue analogue selected from the group consisting of NiHCF, CoHCF, CuHCF, FeHCF, Prussian Blue, MnHCF, ZnHCF, VHCF, CrHCF, MB modified nanoparticles, Fc and derivative nanoparticles, Azure A/C modified nanoparticles, toluidine blue O modified nanoparticles, [Ru(NH₃)₆]^{3+/2+}, [Os(bpy)₃]^{2+/3+}, and cobaltocene.

4. The nanoparticle of claim 1, wherein the redox-active core comprises nickel hexacyanoferrate nanocubes.

5. The nanoparticle of claim 2, wherein the molecularly imprinted polymer comprises a monomer and a crosslinker.

6. The nanoparticle of claim 5, wherein the monomer is a methacrylic acid monomer and the crosslinker is an ethylene glycol dimethacrylate crosslinker.

7. The nanoparticle of claim 1, wherein the target-specific binding cavities are formed by polymerizing one or more monomers in the presence of a template molecule and an initiator, and subsequently removing the template molecule to define the target-specific binding cavities.

8. The nanoparticle of claim 7, wherein the template molecule is selected from the group consisting of ascorbic acid, tryptophan, creatinine, busulfan, cyclophosphamide, mycophenolic acid, glutamate, phenylalanine, cortisol, dopamine, bisphenol A (BPA), aflatoxin B1, glucose, uric acid, lactate, COVID-19 spike protein fragments, and microRNAs.

9. A nanoparticle, comprising:
a core comprising nanocubes; and
a molecularly imprinted polymer shell disposed on the nanocubes, the molecularly imprinted shell comprising a cross-linked copolymer formed in the presence of a template molecule and subjected to template extraction to generate target-selective binding cavities.

10. The nanoparticle of claim 9, wherein the core is a redox-active core.

11. The nanoparticle of claim 9, wherein the nanocubes comprise nickel hexacyanoferrate.

12. The nanoparticle of claim 9, wherein the nanocubes comprises a Prussian blue analogue selected from the group consisting of NiHCF, CoHCF, CuHCF, FeHCF, Prussian Blue, MnHCF, ZnHCF, VHCF, CrHCF, MB modified nanoparticles, Fc and derivative nanoparticles, Azure A/C modified nanoparticles, toluidine blue O modified nanoparticles, [Ru(NH₃)₆]^{3+/2+}, [Os(bpy)₃]^{2+/3+}, and cobaltocene.

13. The nanoparticle of claim 9, wherein the cross-linked copolymer is formed by co-polymerization of methacrylic acid and ethylene glycol dimethacrylate.

14. The nanoparticle of claim 9, wherein the template molecule is selected from the group consisting of ascorbic acid, tryptophan, creatinine, busulfan, cyclophosphamide, mycophenolic acid, glutamate, phenylalanine, cortisol, dopamine, bisphenol A (BPA), aflatoxin B1, glucose, uric acid, lactate, COVID-19 spike protein fragments, and microRNAs.

15. The nanoparticle of claim 9, wherein the target-selective binding cavities are complementary in shape, size, and orientation to the template molecule.

16. A method of manufacturing nanoparticles, comprising:
(a) synthesizing a redox-active core comprising nanocubes by reacting compounds in the presence of a chelating agent;
(b) contacting the nanocubes with a mixture of a monomer, a crosslinker, a template molecule, and an initiator;
(c) polymerizing the mixture to form a molecularly imprinted polymer shell on the nanocubes; and
(d) extracting the template molecule from the molecularly imprinted polymer shell to yield nanoparticles comprising target-selective binding cavities.

17. The method of claim 16, wherein the nanocubes are nickel hexacyanoferrate nanocubes.

18. The method of claim 16, wherein the compounds comprise nickel (II) salt and potassium hexacyanoferrate (III).

19. The method of claim 16, wherein the chelating agent is selected from the group consisting of trisodium citrate dihydrate, EDTA, oxalic acid, tartaric acid, ethylenediamine (en), triethanolamine (TEA), citric acid, ascorbic acid, glutathione, thiol group chelates heavy metals (Au, Cd), nitrilotriacetic acid (NTA), Ni²⁺/Co²⁺, cyanide, Fe(CN)₆]^{3-/4-}, 8-hydroxyquinoline, and dithiocarbonates.

20. The method of claim 16, wherein the monomer is selected from the group consisting of methacrylic acid (MAA), acrylamide (ACM), 4-vinylbenzoic acid (4VB), acrylic acid (AA), itaconic acid, 2-vinylpyridine (2-VP), N-vinylpyrrolidone (NVP), styrene, divinylbenzene (DVB), ethylene glycol dimethacrylate (EGDMA), trimethylolpropane trimethacrylate (TRIM), N,N′-methylenebisacrylamide (MBA), dopamine methacrylamide, boronic acid monomers, 3-acrylamidophenylboronic acid, poly(ethylene glycol) diacrylate (PEGDA), phosphorylcholine methacrylate, N-isopropylacrylamide (NIPAM), and spironaphthoxazine methacrylate.

21. The method of claim 16, wherein the crosslinker is selected from the group consisting of ethylene glycol dimethacrylate (EGDMA), divinylbenzene (DVB), trimethylolpropane trimethacrylate (TRIM), pentaerythritol triacrylate (PETA), poly(ethylene glycol) diacrylate (PEDGA), bisphenol a dimethacrylate (Bis-EMA), N,N′-methylenebisacrylamide (MBA), diallyl tartardiamide (DATD), (3-glycidyloxypropyl)trimethoxysilane (GPTMS), tetraethyl orthosilicate (TEOS), chitosan-glutaraldehyde, genipin, disulfide-bisacrylamide (DSBA), azobenzene dimethacrylate, boronates, 4-vinylphenylboronic acid, metal-organic crosslinkers, and zirconium methacrylate.

22. The method of claim 16, wherein the template molecule is selected from the group consisting of ascorbic acid, tryptophan, creatinine, busulfan, cyclophosphamide, mycophenolic acid, glutamate, phenylalanine, cortisol, dopamine, bisphenol A (BPA), aflatoxin B1, glucose, uric acid, lactate, COVID-19 spike protein fragments, and microRNAs.

23. The method of claim 16, wherein the initiator is selected from the group consisting of azobisisobutyronitrile (AIBN), ammonium persulfate (APS), benzoyl peroxide (BPO), 2,2-dimethoxy-2-phylacetophenone (DMPA), irgacure 2959, eosin Y, tetramethylethylenediamine (APS/TEMED), ferrous sulfate, ferrocenium salts, anthraquinone derivatives, glucose oxidase, horseradish peroxidase (HRP), and biorthogonal.

24. The method of claim 16, wherein step (a) further comprises injecting an aqueous solution comprising nickel (II) acetate and trisodium citrate into an aqueous solution of potassium hexacyanoferrate at a first temperature over a first period of time.

25. The method of claim 24, wherein the polymerization in step (c) is carried out at a second temperature over a second period of time under a nitrogen atmosphere.

26. The method of claim 16, wherein extracting the template molecule comprises washing with a solution comprising an organic solvent.

27. The method of claim 16, further comprising centrifuging and washing the nanoparticles three times with water followed by drying under vacuum.
